(19) United States
(12) Patent Application Publication
(10) Pub. No.: US 2012/0040463 A1
(43) Pub. Date: Feb. 16, 2012

(54) HOLLOW, NOTABLY MULTI-MEMBRANE FIBERS, METHOD FOR PREPARATION THEREOF BY SPINNING AND DEVICE FOR APPLYING SAID METHOD

(75) Inventors: Alain Domard, Lyon (FR); Laurent David, Lyon (FR); Rocío Nohemí Rivas Araiza, Queretaro (MX)

(73) Assignees: CENTRE NATIONAL DE LA RECHERCHE SCIENTIFIQUE, Paris Cedex 16 (FR); UNIVERSITE CLAUDE BERNARD LYON I, Villeurbranne Cedex (FR)

(21) Appl. No.: 12/676,560

(22) PCT Filed: Sep. 5, 2008
(86) PCT No.: PCT/FR2008/051587

§ 371 Date: May 9, 2011

(30) Foreign Application Priority Data
Sep. 7, 2007 (FR) 0757436

(51) Int. Cl. C12N 5/07; B29C 55/22; D01D 5/06

(52) U.S. Cl. 435/400; 264/178 F; 425/66

(57) ABSTRACT

The present invention relates to a method for preparing fibers, notably of polysaccharide or collagen, by wet spinning under coagulation, said method notably comprising: a step for extruding a coagulable macromolecular assembly solution through a normal die; at least one partial coagulation cycle comprising a coagulation step on the one hand and a step for interrupting the coagulation on the other hand; and a step for receiving, notably by winding up, the obtained hollow fiber. The invention also relates to multi-membrane hollow fibers consisting of a same macromolecular assembly which may be spun by coagulation, notably a natural or modified natural polysaccharide in the physical hydrogel or partly dehydrated state. Said fibers including at least over their length two superposed coaxial membranes separated from each other by an inter-membrane space. The invention also relates to the spinning device for applying said method.

This is a 371 national phase application of PCT/FR2008/051587 filed 5 Sep. 2008, claiming priority to French Patent Application No. 0757436 filed 7 Sep. 2007, the contents of which are incorporated herein by reference.

### FIELD OF THE INVENTION

The present invention relates to a method for preparing fibers, notably of polysaccharide or collagen, by wet spinning under coagulation. The invention also relates to hollow fibers, notably consisting of a same natural or modified natural polysaccharide, in the physical hydrogel or partly dehydrated condition, said fibers including at least over their length two superposed coaxial membranes separated from each other by an inter-membrane space. The invention also relates to a spinning device for applying said method.

### BACKGROUND OF THE INVENTION

With the existing methods for preparing hollow fibers of the membrane type, only mono-membrane and bi-membrane systems have been able to be elaborated. The publication of Wang et al. [6] describes bi-membrane hollow fibers, the membranes of which consist of two different polymeric compounds, selected from polysulfone, polyethersulfone, polyetherimide, cellulose acetobutyrate. The publication of Tamura et al. [7] discloses filaments formed with a core of alginate covered with a layer of chitosan, without any inter-membrane space (the numerical references between square brackets relate to the bibliographic references appearing at the end of the description).

Moreover, the elaboration of hollow fibers by wet spinning using an annular die is known, i.e. a die consisting of two concentric cylinders separated by a space allowing a hollow tube to be generated during extrusion. The liquid solution is extruded through the annular space of the die while an internal coagulant agent as a liquid [1-3], gas [4] or compressed air [5] is released inside the extruded tube in order to form the internal wall delimiting the central channel of the fiber, the extruded tube being then immersed in a coagulation bath in order to form the external wall of the fiber.

This spinning technique is complex because of the use of the annular die which requires the introduction of a coagulant agent inside the extruded tube. Further, it is found that with this technique, it is difficult to efficiently control the diameter of the central channel of the fiber.

Further, with this technique for spinning hollow fibers, it is only possible to vary the inner diameter of the fiber, i.e. the diameter of the central channel, by changing the size of the annular die. Further, the thereby obtained fibers have very large inner diameters (350-700 μm), which are not suitable for certain applications [1-2,6], for example in the field of cell cultures or nerve connections, wherein it is often preferable not to exceed an inner diameter of 100 μm.

### SUMMARY OF THE INVENTION

The present invention proposes to find a remedy to the aforementioned drawbacks of the prior art. Its first goal is to provide a method for preparing hollow fibers, notably multi-membrane fibers, simple to apply and with which a variety of hollow fibers may in particular be obtained, which include several coaxial membranes separated from each other by an inter-membrane space. These fibers most often include a central channel, such as known hollow fibers. But they may also not include any central channel, in this case, by extension with respect to the usual terminology, in the present text, they are said to be hollow because of the presence of inter-membrane space(s).

This method is particularly suitable for preparing fibers of polysaccharide or collagen.

For this purpose, the invention according to a first aspect, relates to a method for preparing hollow fibers by wet spinning under coagulation, said method comprising a step

a) for preparing a spinnable solution of a coagulable macromolecular assembly. In a characteristic way, it then comprises:

b) a step for extruding the solution of the coagulable macromolecular assembly through a normal, notably tubular die;

c) at least one partial coagulation cycle, comprising a coagulation step consisting of introducing the extruded solution of the coagulable macromolecular assembly into a coagulation bath containing a coagulation agent on the one hand, by the diffusion of which in said solution it is possible to locally have the macromolecular assembly pass into the coagulated condition, under conditions allowing a fiber to be obtained, the section of which is partly coagulated, and, a step for interrupting coagulation following each coagulation step on the other hand;

d) and a step for receiving, notably by winding, the obtained hollow fiber.

By the expression [symbol] normal die [symbol] is meant here a die which is not annular or which does not apply a central member for forming a central channel in the fiber by internal coagulation, in addition to external coagulation.

By the expression [symbol] spinnable solution [symbol] is meant a solution of a macromolecular assembly, the characteristics of which, notably the flow characteristics, make it suitable for being extruded continuously.

By the expression [symbol] coagulable macromolecular assembly [symbol] , is meant any macromolecule, notably of the polymer or protein type, which may pass from a liquid phase to a solid phase, by the action of a treatment agent, a so-called coagulation agent.

Unlike the existing methods for preparing hollow fibers, the invention neither applies an annular die nor an internal coagulant agent. It is based on voluntary and controlled interruption, during the spinning, of the physico-chemical phenomenon of the external coagulation, for example by a simple washing step with which the coagulant agent may be removed or at least its concentration reduced, thereby stopping the coagulation phenomenon.

If there is only one single partial coagulation step and only one single coagulation interruption step, a mono-membrane hollow fiber is obtained with a central channel, the inner diameter of which is adjustable by varying the conditions of said steps.

As this is to do with the preparation of multi-membrane fibers, the method includes several cycles, each consisting of a partial coagulation step and of a step for interrupting the coagulation. Each cycle allows a coagulated section corresponding to a membrane to be made in the fiber. Further, it was seen that in this case between each cycle, a free space is formed between the two coagulated sections: this is the inter-membrane space.

Thus, with the method according to the invention, it is possible to vary the number of membranes depending on the desired application, by adapting the number of cycles and also the diameter of the die.

Further, as this is to do with the preparation of multi-membrane fibers with a central channel, the present method allows modification of the inner diameter of the central channel, by controlling the interruption of the coagulation during the last cycle, so that the core of the fiber remains as a liquid non-coagulated solution.

By means of the method of the invention, it is possible to obtain mono- or multi-membrane hollow fibers, with the inner diameter of the central channel ranging from the order of 50 μm to beyond 1 mm.

In its general concept, the spinning method of the invention may be applied to all the spinnable macromolecular assemblies under coagulation, including collagen.

In its preferred application, it applies to a wide range of polysaccharides including natural polysaccharides such as chitosan, hyaluronic acid, alginates, pectins, as well as modified natural polysaccharides such as carboxymethylcellulose (CMC).

In this application, the coagulation bath is determined so that its diffusion into the polysaccharide solution allows the saccharide to pass into a physical hydrogel state.

By hydrogel, is meant a visco-elastic mass including at least 80% and preferably at least 90% by mass of water. The hydrogel is said to be physical—as opposed to a so-called chemical hydrogel in which the interactions are of the covalent bond type—when the interactions responsible for the inter-chain cross-linking are of the physical type, notably hydrogen bonds and hydrophobic interactions.

As this is most particularly to do with chitosan, which is a partly or even totally deacetylated derivative of chitin, its acetylation degree (AD) and its molar mass should be taken into account for the coagulable spinnable solution to be applied. The lower the molar mass of chitosan, and the more the chitosan concentration has to be increased into the solution in order to obtain a viscosity making it spinnable, which correlatively leads to adaptation of the coagulation parameters, by increasing the contact times with the coagulation bath and/or the coagulant agent concentration of the coagulation bath. As regards the acetylation degree, it is possible, according to the invention, to use chitosan having an acetylation degree comprised between 0% and 50%, however being aware that the more the AD increases and the lower are the mechanical properties of the coagulated section—therefore of the membrane formed during the coagulation.

Thus, preferably, the spinnable solution is a solution of chitosan having an acetylation degree of less than or equal to 5%, except of course for particular applications where on the contrary, the low rigidity and/or the brittleness of the membranes are required, for example when acceleration of their biodegradation is desired.

Moreover, the object of the invention is to propose multi-membrane hollow fibers of a macromolecular assembly spinnable by coagulation, preferably of polysaccharides, said fibers having a small inner diameter of the central channel and thereby being capable of being used in specific biomedical applications.

Thus, according to a second aspect, the invention relates to a multi-membrane hollow fiber consisting of a same spinnable macromolecular assembly by coagulation, notably of a natural or modified natural polysaccharide, said fiber being characterized in that it includes at least two coaxial membranes, preferably separated from each other by an inter-membrane space.

Preferably, the invention relates to multi-membrane hollow fibers based on polysaccharide, notably on chitosan, the membranes of which are in the physical hydrogel state.

In this preferred alternative, the fibers may be subject to an additional partial dehydration treatment, intended to reduce the water content of hydrogel so as to modulate the rigidity and porosity of the membrane and therefore of the fibers.

The spinning device for applying the method according to the invention is itself also of a simplified design, as compared with the known devices, because the spinning only requires an external coagulation bath type and does not require any internal coagulation bath for forming the central channel or the fiber.

The invention according to a third aspect, relates to a continuous spinning device, specially designed for applying the aforementioned methods, said device comprising:

a) successively the following components:
means for extruding a spinnable solution of a coagulable macromolecular assembly, comprising a normal die,
at least one coagulation reactor intended to contain a coagulation bath, followed by a reactor for interrupting coagulation intended to contain a bath capable of stopping coagulation, notably a washing bath,
and means for winding up the partly coagulated fiber,

b) and means for controlling the coagulation conditions and for interrupting coagulation.

The invention also relates to the use of the aforementioned multi-membrane hollow fibers for elaborating biomaterials.

### BRIEF DESCRIPTION OF THE DRAWINGS

Other characteristics and advantages of the invention will become apparent upon reading the detailed description and exemplary embodiment which will follow, as well as appended figures wherein:

FIG. 1 illustrates a block diagram illustrating the different steps of the continuous spinning method with interrupted coagulation of a multi-membrane hollow fiber of the invention,

FIG. 2 illustrates the structure of a hollow chitosan fiber with four membranes, obtained according to the method of FIG. 1,

FIG. 3 illustrates the macroscopic structure of the fiber of FIG. 2, seen as an enlarged cross-section.

### DETAILED DESCRIPTION

The present invention generally relates to a method for preparing hollow fibers by wet spinning under coagulation, from a spinnable solution of a coagulable macromolecular assembly. In the detailed example which will be described, the question will mainly be a spinnable solution of polysaccharide, most particularly of chitosan, but this should not be restrictive for the present invention. This may also be a spinnable solution of collagen for example.

The solution of polysaccharide is extruded through a normal, notably a tubular die—in any case a die which is not annular—under pressure conditions allowing continuous production of a rod of said solution. By the term of [symbol] rod [symbol] is designated a solid tube as opposed to the hollow tube obtained with an annular die. The relevant rod, formed by the extruded polysaccharide solution is introduced into a coagulation bath, in order to undergo partial coagulation therein.

The coagulation is obtained by the kinetically controlled diffusion of the coagulation agent contained in the coagulation bath, from the outside of the rod, into the polysaccharide solution, with which it is possible to have the polysaccharide locally pass into the physical hydrogel state and obtain a fiber which includes in a cross-section, a partly coagulated area of section.

Coagulation is only partial because a step for interrupting the coagulation necessarily occurs before the coagulation of the polysaccharide solution rod is complete.

Thus, the coagulation reaction is interrupted for example by washing with water in order to obtain on the extruded rod of polysaccharide a coagulated external section forming an external membrane or ‘crown’—in which the polysaccharide is in the physical hydrogel state—which surrounds a ‘core’—in which the polysaccharide is still in the liquid solution state. This first coagulation/washing cycle allows formation of a partially coagulated fiber and therefore a mono-membrane hollow fiber, the central channel of which is obtained by removing the remaining liquid solution by simple washing with water.

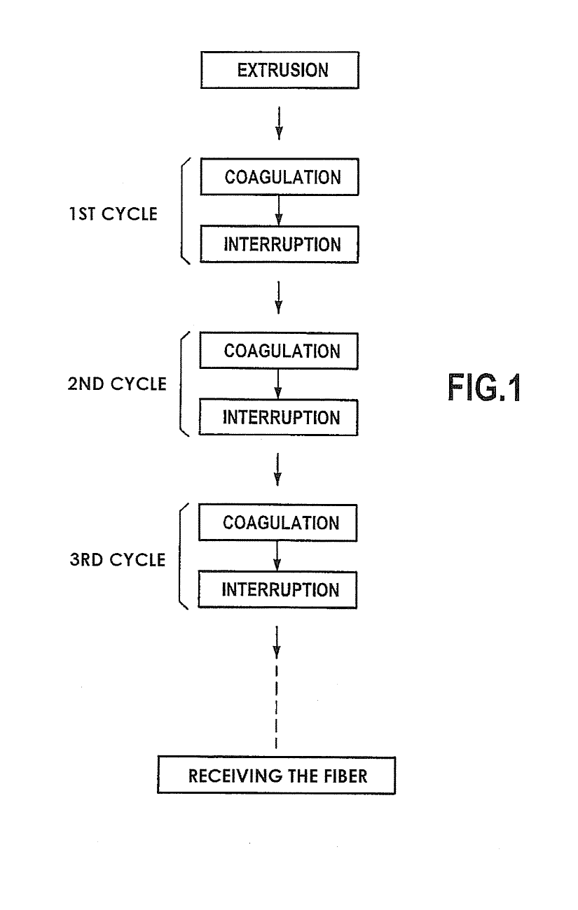

In order to form multi-membrane fibers, according to FIG. 1, the extruded rod of the polysaccharide solution is subject not to only one but to several successive coagulation/washing cycles. During the second cycle, the coagulation bath diffuses through the external membrane and the second additional membrane is foamed while being separated from the external membrane by a free space which forms an inter-membrane space. The same applies cycle after cycle, each new membrane being formed towards the inside of the fiber and being separated from the preceding one by an inter-membrane space.

In order to obtain hollow fibers including a central channel, it is sufficient that the last cycle occurs so that there remains at the core of the fiber a liquid solution of polysaccharide which is finally removed by washing with water.

In an alternative embodiment of the method, coagulation is left to occur until it ends during an ultimate coagulation step, in order to form a fiber with a coagulated core in which the polysaccharide is in the physical hydrogel state. Multi-membrane fibers which are hollow because of the present of the inter-membrane space(s) but which do not include any central channel, may thus be obtained.

It is understood that the operating conditions of each coagulation/washing cycle are the ones which will determine the constitution of each membrane, notably its thickness, and the operating conditions of the successive cycles are the ones which will determine the size of the inter-membrane space separating two adjacent membranes.

For a die with an inner diameter of 1.9 mm, the outer diameter of the fibers may be adjusted between 1.4 and 2.5 mm by varying the output rate at the die outlet. Preferably, for producing multi-membrane hollow fibers, the external diameter of the die is comprised between 1.9 and 2.5 mm.

It is also possible to vary the inner diameter of the central channel of the fibers since this size depends on the final degree of coagulation or on the number and on the thickness of the formed intermediate membranes.

With the method according to the invention, it is possible to obtain mono- or multi-membrane hollow fibers with an inner diameter ranging from 50 μm to beyond 1 mm.

The dwelling time of the extruded polysaccharide solution rod and, then in the subsequent cycles, of the partly coagulated fiber in the coagulation bath and in the washing bath is adjusted by the winding-up rate of the fiber and by the distance which it covers in the corresponding reactors. In order to simplify, the winding-up rate and the concentration of the coagulation bath are kept constant. On the other hand, the distance covered by the fiber in the coagulation and washing reactors may be adjusted by means of turning rolls, adjustable according to different positions.

The method of the invention includes a characterized step for interrupting the coagulation. This may be a simple washing with water of the partly coagulated fiber. This step is essential for forming multi-membrane hollow fibers since the goal is to stop the coagulation reaction, to complete the condensation of chains of polysaccharides into a membrane and to promote the generation of inter-membrane spaces. It is also the fact of stopping the coagulation of the polysaccharide fiber which allows the formation of the central channel of the hollow fiber by stopping coagulation before the setting of the hydrogel of the fiber's core, without it being necessary to preform the cavity by means of a central member with a fixed diameter like in an annular die.

With this sequenced coagulation, it is thereby possible to control the thickness of each membrane as well as the inner diameter of the central channel formed at the core of the fiber. Fibers with very small inner diameters, down to 50 μm, have thus been able to be elaborated by means of the method of the invention.

As compared with known spinning methods, wet spinning with a normal tubular die and with interrupted coagulation, is an advantageous method since it may be applied to a wide range of macromolecular assemblies among which natural or modified natural polysaccharides, such as for example chitosan, hyaluronic acid, alginates, pectins and carboxymethylcellulose (CMC) or further collagen.

When the polysaccharide is chitosan, the latter is dissolved in an aqueous solution of acetic acid and then the thereby obtained chitosan solution is degassed. In a specific exemplary embodiment of a chitosan having an acetylation degree less than or equal to 5% and a molar mass of the order to 500,000 g/mol, the concentration of the chitosan solution should be comprised between 1.5 and 6% by weight. Above 1.5%, the solution is not spinnable, not allowing the extrusion of a continuous chitosan solution rod; above 6%, the viscosity of the solution is too high.

In an alternative embodiment, the method includes a subsequent step for partial dehydration of the obtained fibers, under adaptable conditions with which the water proportion in the hydrogel may be reduced and the rigidity of the membranes and therefore of the fibers may thereby be modulated.

Finally, the method according to the invention also includes a step for receiving, notably by winding up, the obtained hollow fiber.

According to a second aspect, the invention relates to multi-membrane hollow fibers consisting of a same macromolecular assembly spinnable by coagulation, notably a natural or modified natural polysaccharide, said fibers including over the whole of their length and from the outside towards the inside, n coaxial membranes, separated from each other by inter-membrane space, n being an integer greater than or equal to 2.

As this is to do with fibers based on polysaccharide, the latter is in the more or less rigid physical hydrogel state and has porosity comprised between 200 and 500 nm. They may also be partly dried, in order to increase the rigidity and to reduce porosity.

As for the physical structure of the fibers according to the invention, it includes n coaxial membranes, independent of each other and separated from each other by inter-membrane space, which may be comprised between 5 and 20 μm.

Each membrane is delimited by an external face and an internal face. The external face of the fiber is the outermost located external face of the membrane and which forms the crown of the fiber. Similarly, the internal face delimiting the central channel of the hollow fiber is the internal face of the n^{th} membrane, i.e. the one which is the innermost located of the fiber and which is formed during the last coagulation/washing cycle.

The outer diameter of the multi-membrane hollow fibers of the present invention may be of the order of 100 μm up to beyond 2.5 mm. The inner diameter of the central channel of these fibers may be of the order of 50 μm up to beyond 1 mm. The thickness of each membrane may be of the order of 10 μm up to beyond 1 mm.

The polysaccharide is selected from the group: chitosan, hyaluronic acid, alginates, pectins, carboxymethylcellulose.

In a preferred alternative embodiment, the polysaccharide is chitosan. Chitosan, a deacetylated derivative of chitin, is a linear copolymer of β-(1-4) linked D-glucosamine and N-acetyl-D-glucosamine. It is obtained by partial deacetylation of chitin and has the particularity of being soluble in diluted acids, when it is sufficiently deacetylated. This compound is known for its properties of biodegradability, biocompatibility, bioresorbability and bioactivity.

When the polysaccharide making up the fiber according to the invention is chitosan, the fibers in the physical hydrogel state contain 96% of water and 4% of chitosan, before any dehydration treatment.

According to a third aspect, the invention relates to a spinning device for applying the method described earlier, the main elements of which are:
extrusion means capable of forming a solid and continuous rod of a solution of polysaccharide;
at least one coagulation of reactor filled with a coagulant agent and a washing reactor filled with a washed agent in which successively dwells the relevant rod in order to obtain the localized coagulation of the polysaccharide and thereby obtain a partly coagulated polysaccharide fiber;
means for driving and winding up the partly coagulated polysaccharide fiber, said winding means notably comprising a winding motor, a winding spool and rolls;
pumps for renewing the solutions in each reactor;
containers for storing the coagulant agent and the washing agent.

In a specific embodiment of the invention within an experimental laboratory framework, as means for extruding a solution of 1.5 to 6% by weight of chitosan with an AD of less than or equal to 5% in an aqueous solution of acetic acid, use was made of a pump syringe, for example of the BIOBLOCK SCIENTIFIC Model A-99 type and a 20 mL syringe, for example in synthetic polymer, with an outlet diameter corresponding to the die of 1.9 mm. The coagulation reactor contained an aqueous soda solution; the washing reactor contained permutated water; these were two containers with a capacity of about 2.5 L, with a diameter of 12 cm and a height of 25 cm. The means for winding the partly coagulated polysaccharide fiber comprised a winding motor, a winding spool, for example in PVC with a diameter of 4 cm, and turning rolls, for example in PVC with a diameter of 1.3 cm and a length of 8 cm.

In this experimental assembly, the turning rolls gave the possibility of having the fiber successively pass from one bath to the next during the course of each coagulation/washing cycle, their respective location in the reactor allowing adaptation of the dwelling time in the corresponding bath. Industrially, the different cycles are of course carried out continuously and the installation includes means for controlling the conditions of coagulation and interruption of the coagulation, notably controlling the temperature of the baths, the circulation rate of the baths in the reactors, the driving rate of the fiber, the concentration of the coagulation bath and possibly of the bath for interrupting the coagulation if the latter is not a simple bath for washing with water, the extrusion output rate.

### Exemplary Embodiment

### A Hollow Chitosan Fiber with Four Membranes

1. Preparation of the Chitosan Solution

The chitosan used for this work is provided by the Mahtani Chitosan corporation (batch 113). It is derived from squid pens, slightly acetylated (acetylation degree of 1.5%; the acetylation degree represents the number percentage or molar fraction of the N-acetyl glucosamine residues in the chitosan polymer) and has an average mass molar mass of 517,000 g/mol. A 5% (w/w) chitosan spinnable solution is prepared by dissolving chitosan in water and adding acetic acid under stoichiometric conditions relatively to the primary amine functions of chitosan. The thereby obtained solution is degassed.

2. Formation of the Fibers

The following spinning conditions for elaborating fibers were used:
Chitosan solution: 5% (w:w) chitosan
Extrusion rate: 35.4 mL/h
Concentration of the neutralization bath: 0.1M NaOH
Drawing rate: 0.19 cm/s
Volume of coagulant agent in the coagulation reactor: 2.5 L
Volume of permutated water in the washing reactor: 2.5 L.

The extrusion occurs directly in the coagulation bath, the extrusion means and the coagulation reactor being arranged in such a way that there is no air space between the die outlet and the coagulation bath.

3. Formation of Multi-Membrane Fibers—Operating Conditions:
First coagulation bath: 0.10M NaOH
   Coagulation time: 114 s
First bath for washing with water
   Washing time: 60 s
Second coagulation bath: 0.10M NaOH
   Volume of the coagulation bath: 100 mL
   Coagulation time: 60 s
Second bath for washing with water
   Volume of the washing bath: 200 ml
   Washing time: 60 s
Third coagulation bath: 0.10M NaOH
   Volume of the coagulation bath: 100 mL
   Coagulation time: 90 s
Third bath for washing with water
   Volume of the washing bath: 200 ml
   Washing time: 60 s
Fourth coagulation bath: 0.10M NaOH
   Volume of the coagulation bath: 100 mL
   Coagulation time: 120 s
Fourth bath for washing with water
   Volume of the washing bath: 200 ml
   Washing time: 60 s.

Optionally followed by final washing in order to remove the liquid chitosan solution remaining in the central channel of the fiber.

4. Characteristics of the Hollow Multi-Membrane Chitosan Fiber

a) The dimensions of the fiber are:
Outer diameter: 2.5 mm
Inner diameter of the central channel: 1.1 mm
Thickness of each membrane comprised between 200 and 220 μm.

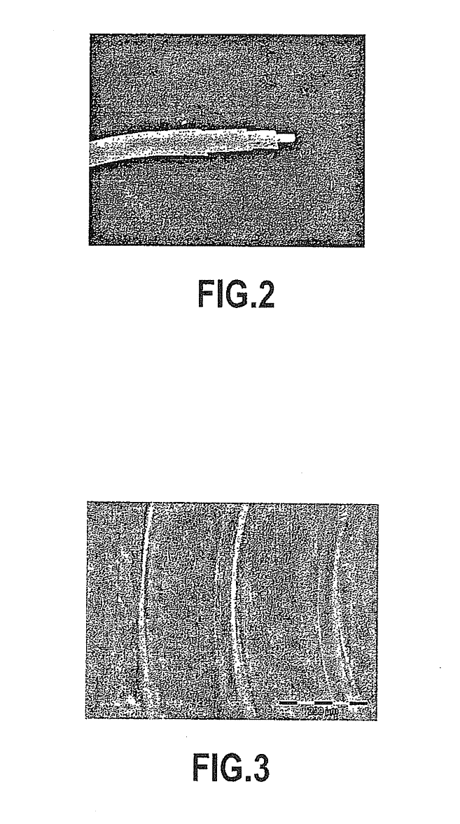

The thereby obtained chitosan fiber with four membranes is illustrated in FIG. 2. FIG. 3 shows the enlarged image of the same fiber seen cross-sectionally.

The hollow multi-membrane fibers according to the invention have many advantages. Fibers including a variable number of membranes may be proposed for each specific application. When they are obtained as a physical hydrogel, they are porous with a pore size between 200 and 500 nm, and may be partly dried in order to form more or less rigid shapes.

The multi-membrane chitosan-based hollow fibers are an excellent candidate for elaborating biomaterials by the biocompatibility, biodegradability, bioactivity and the moderate cost of chitosan.

These fibers may be used as bioreactors for tissue engineering since the multi-membrane system is very well adapted to the regeneration of multilayer tissues with a cylindrical geometry such as blood vessels. The inter-membrane spaces may be colonized with different types of cells, in order to form a cell co-culture system. Thus, in the hollow fibers with four concentric membranes, there are three annular cavities where it is possible to deposit the different types of cells present in blood vessels: endothelial cells, smooth muscle cells and cells of the connective tissue. The low porosity of the membranes makes them impermeable to cells colonizing the bioreactor, which cannot diffuse there through.

Other applications of hollow multi-membrane fibers according to the invention relate to their use for elaborating complex bioreactors for co-cultivating different types of cells and/or for controlled release of different types of active ingredients optionally in combination with the cells.

### BIBLIOGRAPHIC REFERENCES

1. Liu C, Bai R. Preparation of chitosan/cellulose acetate blend hollow fibers for adsorptive perfamiance. J. Membr. Sci. 2005; 267: 68-77.
2. Modrzejewska Z, Eckstein W. Chitosan hollow fiber membranes. Biopolymers 2004; 73:61-68.
3. Qin J-J, Gu J, Chung T-S. Effect of wet and dry-jet wet spinning on the shear induced orientation during the formation of ultrafiltration hollow fiber membranes. J. Membr. Sci. 2001; 182: 57-75.
4. Pittalis F, Bartoli F, Giovannoni G. Process for the preparation of chitosan fibers. U.S. Pat. No. 4,464,321, 1984.
5. Vincent T, Guibal E. Cr (VI) Extraction using aliquat 336 in a hollow fiber module made of chitosan. Ing. Eng. Chem. Res. 2001; 40: 1406-1411.
6. Wang D, Li K, Teo WK. Preparation of annular hollow fibre membranes. J. Membr. Sci. 2000; 166: 31-39.
7. Tamura H, Tsuruta Y, Tokura S. Preparation of chitosan-coated alginate filament. Mater. Sci. Eng. C 2002; 20: 143-147

1. A method for preparing a hollow fiber by wet spinning under coagulation, comprising:
a) preparing a spinnable solution of a coagulable macromolecular assembly,
b) extruding the coagulable macromolecular assembly solution through a normal, notably tubular die;
c) at least one partial coagulation cycle comprising performing a coagulation procedure on the one hand comprising introducing the extruded solution of the coagulable macromolecular assembly into a coagulation bath containing a coagulant agent, the diffusion of which in the solution allows the macromolecular assembly to locally pass into the coagulated state, under conditions allowing a fiber to be obtained which, cross-sectionally, has a partly coagulated section and interrupting the coagulation on the other hand;
d) and receiving, notably by winding up, the obtained hollow fiber.

2. The method according to claim 1 for preparing a mono-membrane hollow fiber wherein the method includes a single partial coagulation cycle.

3. The method according to claim 1 for preparing a multi-membrane hollow fiber with n membranes, wherein the method includes n successive cycles of partial coagulation, n being an integer equal to or greater than 2.

4. The method according to claim 2 for preparing a hollow fiber with a central channel, wherein the method further comprises removing, notably by washing, the solution remaining in the non-coagulated central section of the fiber at the end of the last partial coagulation cycle.

5. The method according to claim 3 for preparing a multi-membrane hollow fiber without any central channel, wherein the method further comprises coagulating the solution remaining in the non-coagulated central section after the last partial coagulation cycle.

6. The method according to claim 1, wherein the spinnable solution is a solution of polysaccharide, notably selected from the group formed by chitosan, hyaluronic acid, alginates, pectins, carboxymethylcellulose and in wherein the coagulant agent allows the polysaccharide to pass into the physical hydrogel state.

7. The method according to claim 6, wherein the coagulation interruption comprises a simple washing with water of the partially coagulated fiber.

8. The method according to claim 6, wherein, as the spinnable solution is a solution of a chitosan having an acetylation degree less than or equal to 5% and a molar mass of the order 500,000 g/mol, the preparation of the spinnable solution comprises the dissolution of said chitosan in an aqueous acetic acid solution in order to obtain a concentration from 1.5 to 6% by weight of chitosan, and then degassing of the latter.

9. The method according to claim 6, further comprising partial dehydration of the obtained fiber, under adaptable conditions allowing reduction of the water proportion in the physical hydrogel.

10. A multi-membrane hollow fiber which comprises a same macromolecular assembly, spinnable by coagulation, and which includes at least two coaxial membranes, preferably separated from each other by an inter-membrane space.

11. The multi-membrane hollow fiber according to claim 10 wherein the macromolecular assembly is a polysaccharide in the physical hydrogel state.

12. The multi-membrane hollow fiber according to claim 11 wherein the polysaccharide is selected from the group: chitosan, hyaluronic acid, alginates, pectins, carboxymethylcellulose.

13. The multi-membrane hollow fiber according to claim 10 wherein the macromolecular assembly is collagen.

14. The multi-membrane hollow fiber according to claim 10 including the following characteristics: an outer diameter of the order of 100 μm to 2.5 mm, a thickness of the membranes of the order of 10 μm to 1 mm, and optionally an inner diameter of the order of 50 μm to 1 mm.

15. The multi-membrane hollow fiber according to claim 14 having an outer diameter of the order of 2.5 mm, a thickness of the membranes of 200 to 220 μm, an inner diameter of the order of 1.1 mm.

16. The multi-membrane hollow fiber according to claim 11 having a porosity comprised between 200 and 500 nm.

17. The multi membrane of chitosan according to claim 12, the physical hydrogel of which contains of the order of 4% of chitosan and of the order of 96% of water.

18. The multi-membrane hollow fiber according to claim 12 wherein the physical hydrogel is in a partial dehydration state.

19. The use of a hollow fiber according to claim 10 for elaborating biomaterials.

20. The use according to claim 19 for elaborating bioreactors for tissue engineering.

21. The use according to claim 19 for elaborating mixed bioreactors with controlled desalting of active ingredients or of macromolecules of biological interest.

22. The use according to claim 19 for elaborating membranes of biological interest.

23. A continuous spinning device for applying the method for preparing a hollow fiber from a macromolecular assembly according to claim 1, said device comprising:
a. a device that extrudes a spinnable solution of coagulable macromolecule assembly, comprising a normal die;
b. at least one partial coagulation assembly comprising a coagulation reactor intended for containing a coagulation bath containing a coagulant agent on the one hand and a reactor for interrupting coagulation intended for containing a bath capable of stopping coagulation on the other hand;
c. a device that winds up the fiber;
d. a device that controls the coagulation and coagulation interruption conditions.

24. The use of a hollow fiber obtained by the method according to claim 1 for elaborating biomaterials.
